(19) United States
(12) Patent Application Publication
(10) Pub. No.: US 2015/0299807 A1
(43) Pub. Date: Oct. 22, 2015

(54) GENOMIC CLASSIFIERS FOR NON-INVASIVE IDENTIFICATION OF HIGH GRADE PROSTATE CANCER WITH METASTATIC POTENTIAL

(71) Applicant: THE JOHNS HOPKINS UNIVERSITY, Baltimore, MD (US)

(72) Inventors: Ashley E. ROSS, Columbia, MD (US); Edward M. SCHAEFFER, Sparks, MD (US)

(21) Appl. No.: 14/646,427

(22) PCT Filed: Nov. 21, 2013
(86) PCT No.: PCT/US2013/071206

§ 371 Date: May 21, 2015

Related U.S. Application Data
(60) Provisional application No. 61/728,957, filed on Nov. 21, 2012.

(51) Int. Cl. C12Q 1/68; A61B 5/15; A61B 10/00; G01N 33/574

(52) U.S. Cl. CPC C12Q 1/6886; G01N 33/57434; A61B 5/15; A61B 10/007; A61B 10/0038; A61B 10/0058; G01N 2800/56; C12Q 2600/158; C12Q 2600/112; H01J 49/0036

(57) ABSTRACT

The present invention relates to the field of biomarkers. More specifically, the present invention provides methods and compositions useful for diagnosing and/or prognosing prostate cancer. In a specific embodiment, a method for diagnosing prostate cancer or a likelihood thereof in a patient comprising the steps of (a) obtaining a biological sample from the patient; (b) subjecting the sample to an assay for detecting expression of one or more of ACSM2A, BDH2, C19orf51, C8orf76, CGB5, CSMD3, DAZ2, DUX4, FAM22G, FAM90A1, GABBR2, GRM3, HMMR, HOXC4, KAAG1, KRIT1, KRTAP20-1, LOC392196, LOC441956, LOC650293, LTB4R, METTL7B, NEK2, OR11H12, OR2J3, OR2L8, OR2M1P, OR2T3, OR4F5, OR52A4, OR5211, PGA3, PHACTR3, PMP2, PRAMEF6, PSG1, SIGLEC10, SOX11, SPDYE1, SSX1, TCEB3B, TCFL5, TFAP2D, TSPY2, UGT2B10, UGT2B11, UGT2B28, WDR49, and WFDC5; and (c) determining that the patient has prostate cancer or a likelihood thereof if the expression of the one or more biomarkers is increased relative to a reference non-prostate cancer sample.

### CROSS-REFERENCE TO RELATED APPLICATIONS

This application claims the benefit of U.S. Provisional Application No. 61/728,957, filed Nov. 21, 2012; which is incorporated herein by reference in its entirety.

### FIELD OF THE INVENTION

The present invention relates to the field of biomarkers. More specifically, the present invention provides methods and compositions useful for diagnosing and/or prognosing prostate cancer.

### BACKGROUND OF THE INVENTION

Current prostate cancer screening relies on prostate serum antigen (PSA) testing and clinical staging by digital rectal exam (DRE). Though widespread use of PSA screening has resulted in the earlier detection of prostate cancer, screening in this fashion carries with it valid concerns for the over use of invasive diagnostic procedures (prostate biopsy) and the subsequent over-diagnosis and over-treatment of prostate cancer. This stems primarily from the substantial prevalence of clinically indolent prostate cancer and the inability of non-invasive screening methods to identify life threatening or clinically significant disease. Accordingly, better methods for prostate cancer screening are needed.

### SUMMARY OF THE INVENTION

The present invention is based, at least in part, on the discovery of an expression signature unique to high grade prostate cancer with metastatic potential. The present inventors discovered the signature by comparing the genomic expression profiles of high grade prostate cancer with rapid metastasis after local treatment to non-metastatic prostate cancers and benign prostate and urogenital tissue. The present invention can be used in non-invasive urine and serum based diagnostic assays.

By employing methods for genome wide expression analysis from minimal amounts of routinely stored pathological tissue, the present inventors were able to molecularly characterize prostate cancer from individuals with prostate cancer and known, disparate clinical outcomes. Further, the classifier described herein is based on the cancers of a unique set of men who had aggressive localized disease at diagnosis but no neoadjuvant treatment prior to surgery and who lacked adjuvant treatment prior to the development of metastasis. This fundamentally differs from genomic classifiers which may predict cancer aggressiveness but are not prostate cancer specific including, for example, the Prolaris test by Myriad Genetics.

Accordingly, in one aspect, the present invention provides methods for diagnosing high grade prostate cancer with metastatic potential in a patient. In one embodiment, the method comprises the steps of (a) obtaining a biological sample from the patient; (b) quantitating the biomarker expression levels of one or more of ACSM2A, BDH2, C19orf51, C8orf76, CGB5, CSMD3, DAZ2, DUX4, FAM22G, FAM90A1, GABBR2, GRM3, HMMR, HOXC4, KAAG1, KRIT1, KRTAP20-1, LOC392196, LOC441956, LOC650293, LTB4R, METTL7B, NEK2, OR11H12, OR2J3, OR2L8, OR2M1P, OR2T3, OR4F5, OR52A4, OR5211, PGA3, PHACTR3, PMP2, PRAMEF6, PSG1, SIGLEC10, SOX11, SPDYE1, SSX1, TCEB3B, TCFL5, TFAP2D, TSPY2, UGT2B10, UGT2B11, UGT2B28, WDR49, and WFDC5; (c) comparing the levels of the one or more biomarkers with reference levels of the one or more biomarkers that correlate to a patient not having prostate cancer with metastatic potential; and (d) identifying the patient as having prostate cancer with metastatic potential if the quantitated amounts of the one or more biomarkers is increased compared to the reference levels. The sample can be any biological sample including blood, plasma, serum, urine, stool or semen. In a specific embodiment, the sample is a urine sample. In another specific embodiment, the sample is a semen sample. In a further specific embodiment, the sample is a serum sample.

In certain embodiments, the quantitation step comprises performing multiplex quantitative real-time polymerase chain reaction. In other embodiments, the patient not having prostate cancer with metastatic potential comprises one or more of patients with low grade prostate cancer, high grade prostate cancer without metastatic potential, normal prostate epithelium, benign prostate hyperplasia and benign urothelium. In further embodiments, the levels of the one or more biomarkers from the patient sample are increased at least 4-fold as compared to reference levels of the same biomarkers.

The present invention also provides a method for diagnosing high grade prostate cancer with metastatic potential in a patient comprising the steps of (a) obtaining a biological sample from the patient; (b) subjecting the sample to an assay for detecting expression of one or more of ACSM2A, BDH2, C19orf51, C8orf76, CGB5, CSMD3, DAZ2, DUX4, FAM22G, FAM90A1, GABBR2, GRM3, HMMR, HOXC4, KAAG1, KRIT1, KRTAP20-1, LOC392196, LOC441956, LOC650293, LTB4R, METTL7B, NEK2, OR11H12, OR2J3, OR2L8, OR2M1P, OR2T3, OR4F5, OR52A4, OR5211, PGA3, PHACTR3, PMP2, PRAMEF6, PSG1, SIGLEC10, SOX11, SPDYE1, SSX1, TCEB3B, TCFL5, TFAP2D, TSPY2, UGT2B10, UGT2B11, UGT2B28, WDR49, and WFDC5; and (c) comparing the levels of the one or more biomarkers with predefined levels of the same biomarkers that correlate to a patient having high grade prostate cancer with metastatic potential and predefined levels of the same biomarkers that correlate to a patient not having high grade prostate cancer with metastatic potential, wherein a correlation to one of the predefined levels provides the diagnosis. The sample can be any biological sample including blood, plasma, serum, urine, stool or semen. In a specific embodiment, the sample is a urine sample. In another specific embodiment, the sample is a semen sample. In a further specific embodiment, the sample is a serum sample. In certain embodiments, the assay for detecting expression is an immunoassay. In other embodiments, the assay for detecting expression is mass spectrometry. In a specific embodiment, the mass spectrometry is multiple reaction monitoring mass spectrometry (MRM-MS).

In an alternative embodiment, a method for diagnosing high grade prostate cancer with metastatic potential in a patient comprises the steps of (a) obtaining a sample from a patient suspected of having prostate cancer; (b) quantitating the amount of the one or more biomarkers listed in Table 1, wherein the quantitating step comprises (i) contacting the sample with a set of primers capable of amplifying one or more of the biomarkers listed in Table 1; and (ii) amplifying the one or more biomarkers listed in Table 1; (c) comparing the quantitated amounts of the one or more biomarkers listed in Table 1 to a reference level; and (d) identifying the patient as having prostate cancer if the quantitated amounts of the one or more biomarkers is increased compared to the reference level.

In another aspect, the present invention provides methods of treatment. In one embodiment, a method for treating a patient suspected of having or likely to develop high grade prostate cancer with metastatic potential comprises the steps of (a) obtaining a biological sample from the patient; (b) quantitating the biomarker expression levels of one or more of ACSM2A, BDH2, C19orf51, C8orf76, CGB5, CSMD3, DAZ2, DUX4, FAM22G, FAM90A1, GABBR2, GRM3, HMMR, HOXC4, KAAG1, KRIT1, KRTAP20-1, LOC392196, LOC441956, LOC650293, LTB4R, METTL7B, NEK2, OR11H12, OR2J3, OR2L8, OR2M1P, OR2T3, OR4F5, OR52A4, OR52I1, PGA3, PHACTR3, PMP2, PRAMEF6, PSG1, SIGLEC10, SOX11, SPDYE1, SSX1, TCEB3B, TCFL5, TFAP2D, TSPY2, UGT2B10, UGT2B11, UGT2B28, WDR49, and WFDC5; (c) comparing the levels of the one or more biomarkers with reference levels of the one or more biomarkers that correlate to a patient not having prostate cancer with metastatic potential; (d) identifying the patient as having or likely to develop prostate cancer with metastatic potential if the quantitated amounts of the one or more biomarkers is increased compared to the reference levels; and (e) performing prostatectomy on the patient. The sample can be any biological sample including blood, plasma, serum, urine, stool or semen.

In yet another aspect, the present invention provides methods for diagnosing prostate cancer in a patient. In one embodiment, a method for diagnosing prostate cancer or a likelihood thereof in a patient comprises the steps of (a) obtaining a biological sample from the patient; (b) subjecting the sample to an assay for detecting expression of one or more biomarkers listed in Table 1; and (c) determining that the patient has prostate cancer or a likelihood thereof if the expression of the one or more biomarkers is increased relative to a reference non-prostate cancer sample.

In another method, a method for identifying prostate cancer lesions with metastatic potential in a patient comprises the steps of (a) obtaining a biological sample from the patient; (b) subjecting the sample to an assay for detecting expression of one or more biomarkers listed in Table 1; and (c) determining that the cancer lesions have metastatic potential if the expression of the one or more biomarkers is increased relative to a reference non-prostate cancer sample.

In a further embodiment, a method for predicting metastasis in a prostate cancer patient comprises the steps of (a) obtaining a biological sample from the patient; (b) subjecting the sample to an assay for detecting expression of one or more biomarkers listed in Table 1; and (c) determining that metastasis is likely to occur if the expression of the one or more biomarkers is increased relative to a reference non-prostate cancer sample.

In an alternative embodiment, a method for determining a likelihood of prostate cancer recurrence in a patient following prostatectomy comprises the steps of (a) obtaining a biological sample from the patient; (b) subjecting the sample to an assay for detecting expression of one or more biomarkers listed in Table 1; and (c) determining that prostate cancer is likely to recur if the expression of the one or more biomarkers is increased relative to a reference non-prostate cancer sample.

In another embodiment, a method for determining a likelihood of prostate cancer recurrence in a patient following prostatectomy comprises the steps of (a) obtaining a prostate tissue sample from the patient; (b) subjecting the sample to an assay for detecting expression of one or more biomarkers listed in Table 1; (c) providing a reference non-prostate cancer tissue sample; (d) comparing the level of expression of the one or more biomarkers from the prostate tissue sample of the patient to the level of expression of the same biomarkers in the reference non-prostate cancer tissue sample; and (e) determining that prostate cancer is likely to recur when the level of expression of the one or more biomarkers in the prostate tissue sample of the patient is increased relative to the level of expression of the one or more biomarkers in the reference non-prostate cancer tissue sample.

In a more specific embodiment, a method for diagnosing prostate cancer or a likelihood thereof in a patient comprises the steps of (a) obtaining a biological sample from the patient; (b) subjecting the sample to an assay for detecting expression of one or more of ACSM2A, BDH2, C19orf51, C8orf76, CGB5, CSMD3, DAZ2, DUX4, FAM22G, FAM90A1, GABBR2, GRM3, HMMR, HOXC4, KAAG1, KRIT1, KRTAP20-1, LOC392196, LOC441956, LOC650293, LTB4R, METTL7B, NEK2, OR11H12, OR2J3, OR2L8, OR2M1P, OR2T3, OR4F5, OR52A4, OR5211, PGA3, PHACTR3, PMP2, PRAMEF6, PSG1, SIGLEC10, SOX11, SPDYE1, SSX1, TCEB3B, TCFL5, TFAP2D, TSPY2, UGT2B10, UGT2B11, UGT2B28, WDR49, and WFDC5; and (c) determining that the patient has prostate cancer or a likelihood thereof if the expression of the one or more biomarkers is increased relative to a reference non-prostate cancer sample.

In another embodiment, a method for diagnosing high grade prostate cancer with metastatic potential in a patient comprises the steps of (a) obtaining a biological sample from the patient; (b) subjecting the sample to an assay for detecting expression of one or more of ACSM2A, BDH2, C19orf51, C8orf76, CGB5, CSMD3, DAZ2, DUX4, FAM22G, FAM90A1, GABBR2, GRM3, HMMR, HOXC4, KAAG1, KRIT1, KRTAP20-1, LOC392196, LOC441956, LOC650293, LTB4R, METTL7B, NEK2, OR11H12, OR2J3, OR2L8, OR2M1P, OR2T3, OR4F5, OR52A4, OR5211, PGA3, PHACTR3, PMP2, PRAMEF6, PSG1, SIGLEC10, SOX11, SPDYE1, SSX1, TCEB3B, TCFL5, TFAP2D, TSPY2, UGT2B10, UGT2B11, UGT2B28, WDR49, and WFDC5; and (c) comparing the levels of the one or more biomarkers with predefined levels of the same biomarkers that correlate to a patient having high grade prostate cancer with metastatic potential and predefined levels of the same biomarkers that correlate to a patient not having high grade prostate cancer with metastatic potential, wherein a correlation to one of the predefined levels provides the diagnosis.

In the methods of the present invention, the sample is blood, plasma, serum, urine, stool or semen. In a specific embodiment, the sample is a urine sample. In another embodiment, the sample is a semen sample. In yet another embodiment, the sample is a serum sample.

In a specific embodiment, the assay for detecting expression is an immunoassay. In an alternative embodiment, the assay for detecting expression is mass spectrometry. In a more specific embodiment, the mass spectrometry is multiple reaction monitoring mass spectrometry (MRM-MS).

In a further embodiment, the present invention provides a prostate cancer genomic classifier comprising one or more biomarkers listed in Table 1.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

As used herein, the term “comparing” refers to making an assessment of how the proportion, level or cellular localization of one or more biomarkers in a sample from a patient relates to the proportion, level or cellular localization of the corresponding one or more biomarkers in a standard or control sample. For example, “comparing” may refer to assessing whether the proportion, level, or cellular localization of one or more biomarkers in a sample from a patient is the same as, more or less than, or different from the proportion, level, or cellular localization of the corresponding one or more biomarkers in standard or control sample. More specifically, the term may refer to assessing whether the proportion, level, or cellular localization of one or more biomarkers in a sample from a patient is the same as, more or less than, different from or otherwise corresponds (or not) to the proportion, level, or cellular localization of predefined biomarker levels/ratios that correspond to, for example, a patient having prostate cancer, not having prostate cancer, is responding to treatment for prostate cancer, is not responding to treatment for prostate cancer, is/is not likely to respond to a particular prostate cancer treatment, or having /not having another disease or condition. In a specific embodiment, the term “comparing” refers to assessing whether the level of one or more biomarkers of the present invention in a sample from a patient is the same as, more or less than, different from other otherwise correspond (or not) to levels/ratios of the same biomarkers in a control sample (e.g., predefined levels/ratios that correlate to uninfected individuals, standard prostate cancer levels/ratios, etc.).

In another embodiment, the term “comparing” refers to making an assessment of how the proportion, level or cellular localization of one or more biomarkers in a sample from a patient relates to the proportion, level or cellular localization of another biomarker in the same sample. For example, a ratio of one biomarker to another from the same patient sample can be compared. In another embodiment, a level of one biomarker in a sample (e.g., a post-translationally modified biomarker protein) can be compared to the level of the same biomarker (e.g., unmodified biomarker protein) in the sample. Ratios of modified:unmodified biomarker proteins can be compared to other protein ratios in the same sample or to predefined reference or control ratios.

As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient has prostate cancer. In specific embodiments, the parameter may comprise the level of one or more biomarkers of the present invention. A particular set or pattern of the amounts of one or more biomarkers may indicate that a patient has prostate cancer (i.e., correlates to a patient having prostate cancer). In other embodiments, a correlation could be the ratio of a post-translationally modified protein to the unmodified protein indicates (or a change in the ratio over time or as compared to a reference/control ratio) could mean that the patient has prostate cancer). In specific embodiments, a correlation could be the ratio of modified protein to the unmodified protein, or any other combination in which a change in one protein causes or is accompanied by a change in another.

In other embodiments, a particular set or pattern of the amounts of one or more biomarkers may be correlated to a patient being unaffected (i.e., indicates a patient does not have prostate cancer). In certain embodiments, “indicating,” or “correlating,” as used according to the present invention, may be by any linear or non-linear method of quantifying the relationship between levels/ratios of biomarkers to a standard, control or comparative value for the assessment of the diagnosis, prediction of prostate cancer or prostate cancer progression, assessment of efficacy of clinical treatment, identification of a patient that may respond to a particular treatment regime or pharmaceutical agent, monitoring of the progress of treatment, and in the context of a screening assay, for the identification of an anti-prostate cancer therapeutic.

The terms “patient,” “individual,” or “subject” are used interchangeably herein, and refer to a mammal, particularly, a human. The patient may have a mild, intermediate or severe disease or condition. The patient may be treatment naïve, responding to any form of treatment, or refractory. The patient may be an individual in need of treatment or in need of diagnosis based on particular symptoms or family history. In certain embodiments, the term patient refers to a fetus or a neonate. In some cases, the terms may refer to treatment in experimental animals, in veterinary application, and in the development of animal models for disease, including, but not limited to, rodents including mice, rats, and hamsters; and primates.

The terms “measuring” and “determining” are used interchangeably throughout, and refer to methods which include obtaining or providing a patient sample and/or detecting the level of a biomarker(s) in a sample. In one embodiment, the terms refer to obtaining or providing a patient sample and detecting the level of one or more biomarkers in the sample. In another embodiment, the terms “measuring” and “determining” mean detecting the level of one or more biomarkers in a patient sample. Measuring can be accomplished by methods known in the art and those further described herein. The term “measuring” is also used interchangeably throughout with the term “detecting.” In certain embodiments, the term is also used interchangeably with the term “quantitating.”

The terms “sample,” “patient sample,” “biological sample,” and the like, encompass a variety of sample types obtained from a patient, individual, or subject and can be used in a diagnostic or monitoring assay. The patient sample may be obtained from a healthy subject or a patient having symptoms associated with prostate cancer. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiments, a sample comprises blood. In other embodiments, a sample comprises serum. In a specific embodiment, a sample comprises plasma. In another embodiment, a sample comprises urine. In yet another embodiment, a semen sample is used. In a further embodiment, a stool sample is used.

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

An “antibody” is an immunoglobulin molecule that recognizes and specifically binds to a target, such as a protein, polypeptide, peptide, carbohydrate, polynucleotide, lipid, etc., through at least one antigen recognition site within the variable region of the immunoglobulin molecule. As used herein, the term is used in the broadest sense and encompasses intact polyclonal antibodies, intact monoclonal antibodies, antibody fragments (such as Fab, Fab′, F(ab′)₂, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be one of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

As used herein, the terms “antibody fragments”, “fragment”, or “fragment thereof” refer to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments. In most embodiments, the terms also refer to fragments that binding an antigen of a target molecule (e.g., a biomarker described in Table 1) and can be referred to as “antigen-binding fragments.”

As used herein, “humanized” forms of non-human (e.g., murine) antibodies are chimeric antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues that are not found in the recipient antibody or in the donor antibody. These modifications are generally made to further refine antibody performance In general, the humanized antibody will comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody can also comprise at least a portion of an immunoglobulin constant region (Fc), typically that of a human immunoglobulin. Examples of methods used to generate humanized antibodies are described in U.S. Pat. No. 5,225,539.

The term “human antibody” as used herein means an antibody produced by a human or an antibody having an amino acid sequence corresponding to an antibody produced by a human made using any of the techniques known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

“Hybrid antibodies” are immunoglobulin molecules in which pairs of heavy and light chains from antibodies with different antigenic determinant regions are assembled together so that two different epitopes or two different antigens can be recognized and bound by the resulting tetramer.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc) with the desired specificity, affinity, and capability while the constant regions are homologous to the sequences in antibodies derived from another (usually human) to avoid eliciting an immune response in that species.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation. An antigenic determinant can compete with the intact antigen (i.e., the “immunogen” used to elicit the immune response) for binding to an antibody.

The terms “specifically binds to,” “specific for,” and related grammatical variants refer to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

Various methodologies of the instant invention include a step that involves comparing a value, level, feature, characteristic, property, etc. to a “suitable control,” referred to interchangeably herein as an “appropriate control,” a “control sample,” a “reference” or simply a “control.” A “suitable control,” “appropriate control,” “control sample,” “reference” or a “control” is any control or standard familiar to one of ordinary skill in the art useful for comparison purposes. In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the biomarkers of the present invention may be assayed for levels/ratios in a sample from an unaffected individual (UI) or a normal control individual (NC) (both terms are used interchangeably herein). In another embodiment, a “suitable control” or “appropriate control” is a value, level, feature, characteristic, property, ratio, etc. determined prior to performing a therapy (e.g., prostate cancer treatment) on a patient. In yet another embodiment, a transcription rate, mRNA level, translation rate, protein level/ratio, biological activity, cellular characteristic or property, genotype, phenotype, etc., can be determined prior to, during, or after administering a therapy into a cell, organ, or patient. In a further embodiment, a “suitable control” or “appropriate control” is a predefined value, level, feature, characteristic, property, ratio, etc. A “suitable control” can be a profile or pattern of levels/ratios of one or more biomarkers of the present invention that correlates to prostate cancer, to which a patient sample can be compared. The patient sample can also be compared to a negative control, i.e., a profile that correlates to not having prostate cancer.

### II. Detection of Prostate Cancer Biomarkers

A. Detection by Polymerase Chain Reaction

In certain embodiments, the biomarkers of the present invention can be detected/measure/quantitated by polymerase chain reaction (PCR). In certain embodiments, the present invention contemplates quantitation of one or more biomarkers described herein including ACSM2A, BDH2, C19orf51, C8orf76, CGB5, CSMD3, DAZ2, DUX4, FAM22G, FAM90A1, GABBR2, GRM3, HMMR, HOXC4, KAAG1, KRIT1, KRTAP20-1, LOC392196, LOC441956, LOC650293, LTB4R, METTL7B, NEK2, OR11H12, OR2J3, OR2L8, OR2M1P, OR2T3, OR4F5, OR52A4, OR5211, PGA3, PHACTR3, PMP2, PRAMEF6, PSG1, SIGLEC10, SOX11, SPDYE1, SSX1, TCEB3B, TCFL5, TFAP2D, TSPY2, UGT2B10, UGT2B11, UGT2B28, WDR49, and WFDC5. The one or more biomarkers can be quantitated and the expression can be compared to reference levels.

Overexpression relative to the reference is indicative of cancer. PCR can include quantitative type PCR, such as quantitative, real-time PCR (both singleplex and multiplex). In a specific embodiments, the quantitation steps are carried using quantitative, real-time PCR. One of ordinary skill in the art can design primers that specifically bind and amplify one or more biomarkers described herein using the publicly available sequences thereof.

B. Detection by Immunoassay

In other embodiments, the biomarkers of the present invention can be detected and/or measured by immunoassay Immunoassay requires biospecific capture reagents, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

Although antibodies are useful because of their extensive characterization, any other suitable agent (e.g., a peptide, an aptamer, or a small organic molecule) that specifically binds a biomarker of the present invention is optionally used in place of the antibody in the above described immunoassays. For example, an aptamer that specifically binds a biomarker and/or one or more of its breakdown products might be used. Aptamers are nucleic acid-based molecules that bind specific ligands. Methods for making aptamers with a particular binding specificity are known as detailed in U.S. Pat. No. 5,475,096; U.S. Pat. No. 5,670,637; U.S. Pat. No. 5,696,249; U.S. Pat. No. 5,270,163; U.S. Pat. No. 5,707,796; U.S. Pat. No. 5,595,877; U.S. Pat. No. 5,660,985; U.S. Pat. No. 5,567,588; U.S. Pat. No. 5,683,867; U.S. Pat. No. 5,637,459; and U.S. Pat. No. 6,011,020.

C. Detection by Electrochemicaluminescent Assay

In several embodiments, the biomarker biomarkers of the present invention may be detected by means of an electrochemicaluminescent assay developed by Meso Scale Discovery (Gaithersrburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. No. 7,497,997; U.S. Pat. No. 7,491,540; U.S. Pat. No. 7,288,410; U.S. Pat. No. 7,036,946; U.S. Pat. No. 7,052,861; U.S. Pat. No. 6,977,722; U.S. Pat. No. 6,919,173; U.S. Pat. No. 6,673,533; U.S. Pat. No. 6,413,783; U.S. Pat. No. 6,362,011; U.S. Pat. No. 6,319,670; U.S. Pat. No. 6,207,369; U.S. Pat. No. 6,140,045; U.S. Pat. No. 6,090,545; and U.S. Pat. No. 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

D. Detection by Mass Spectrometry

In one aspect, the biomarkers of the present invention may be detected by mass spectrometry, a method that employs a mass spectrometer to detect gas phase ions. Examples of mass spectrometers are time-of-flight, magnetic sector, quadrupole filter, ion trap, ion cyclotron resonance, Orbitrap, hybrids or combinations of the foregoing, and the like.

In particular embodiments, the biomarkers of the present invention are detected using selected reaction monitoring (SRM) mass spectrometry techniques. Selected reaction monitoring (SRM) is a non-scanning mass spectrometry technique, performed on triple quadrupole-like instruments and in which collision-induced dissociation is used as a means to increase selectivity. In SRM experiments two mass analyzers are used as static mass filters, to monitor a particular fragment ion of a selected precursor ion. The specific pair of mass-over-charge (m/z) values associated to the precursor and fragment ions selected is referred to as a “transition” and can be written as parent m/z→fragment m/z (e.g. 673.5→534.3). Unlike common MS based proteomics, no mass spectra are recorded in a SRM analysis. Instead, the detector acts as counting device for the ions matching the selected transition thereby returning an intensity distribution over time. Multiple SRM transitions can be measured within the same experiment on the chromatographic time scale by rapidly toggling between the different precursor/fragment pairs (sometimes called multiple reaction monitoring, MRM). Typically, the triple quadrupole instrument cycles through a series of transitions and records the signal of each transition as a function of the elution time. The method allows for additional selectivity by monitoring the chromatographic coelution of multiple transitions for a given analyte. The terms SRM/MRM are occasionally used also to describe experiments conducted in mass spectrometers other than triple quadrupoles (e.g. in trapping instruments) where upon fragmentation of a specific precursor ion a narrow mass range is scanned in MS2 mode, centered on a fragment ion specific to the precursor of interest or in general in experiments where fragmentation in the collision cell is used as a means to increase selectivity. In this application the terms SRM and MRM or also SRM/MRM can be used interchangeably, since they both refer to the same mass spectrometer operating principle. As a matter of clarity, the term MRM is used throughout the text, but the term includes both SRM and MRM, as well as any analogous technique, such as e.g. highly-selective reaction monitoring, hSRM, LC-SRM or any other SRM/MRM-like or SRM/MRM-mimicking approaches performed on any type of mass spectrometer and/or, in which the peptides are fragmented using any other fragmentation method such as e.g. CAD (collision-activated dissociation (also known as CID or collision-induced dissociation), HCD (higher energy CID), ECD (electron capture dissociation), PD (photodissociation) or ETD (electron transfer dissociation).

In another specific embodiment, the mass spectrometric method comprises matrix assisted laser desorption/ionization time-of-flight (MALDI-TOF MS or MALDI-TOF). In another embodiment, method comprises MALDI-TOF tandem mass spectrometry (MALDI-TOF MS/MS). In yet another embodiment, mass spectrometry can be combined with another appropriate method(s) as may be contemplated by one of ordinary skill in the art. For example, MALDI-TOF can be utilized with trypsin digestion and tandem mass spectrometry as described herein.

In an alternative embodiment, the mass spectrometric technique comprises surface enhanced laser desorption and ionization or “SELDI,” as described, for example, in U.S. Pat. No. 6,225,047 and U.S. Pat. No. 5,719,060. Briefly, SELDI refers to a method of desorption/ionization gas phase ion spectrometry (e.g. mass spectrometry) in which an analyte (here, one or more of the biomarkers) is captured on the surface of a SELDI mass spectrometry probe. There are several versions of SELDI that may be utilized including, but not limited to, Affinity Capture Mass Spectrometry (also called Surface-Enhanced Affinity Capture (SEAC)), and Surface-Enhanced Neat Desorption (SEND) which involves the use of probes comprising energy absorbing molecules that are chemically bound to the probe surface (SEND probe). Another SELDI method is called Surface-Enhanced Photolabile Attachment and Release (SEPAR), which involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker panel, pursuant to the present invention.

In another mass spectrometry method, the biomarkers can be first captured on a chromatographic resin having chromatographic properties that bind the biomarkers. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

E. Other Methods for Detecting Biomarkers

The biomarkers of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Invitrogen Corp. (Carlsbad, Calif.), Affymetrix, Inc. (Fremong, Calif.), Zyomyx (Hayward, Calif.), R&D Systems, Inc. (Minneapolis, Minn.), Biacore (Uppsala, Sweden) and Procognia (Berkshire, UK). Examples of such protein biochips are described in the following patents or published patent applications: U.S. Pat. No. 6,537,749; U.S. Pat. No. 6,329,209; U.S. Pat. No. 6,225,047; U.S. Pat. No. 5,242,828; PCT International Publication No. WO 00/56934; and PCT International Publication No. WO 03/048768.

### III. Determination of a Patient's Prostate Cancer Status

A. The present invention relates to the use of biomarkers to diagnose prostate cancer. More specifically, the biomarkers of the present invention can be used in diagnostic tests to determine, qualify, and/or assess prostate cancer or status, for example, to diagnose prostate cancer, in an individual, subject or patient. In particular embodiments, prostate cancer status can include determining a patient's prostate cancer status or prostate cancer status, for example, to diagnose prostate cancer, in an individual, subject or patient. More specifically, the biomarkers to be detected in diagnosing prostate cancer (e.g., high grade prostate cancer) include, but are not limited to, ACSM2A, BDH2, C19orf51, C8orf76, CGB5, CSMD3, DAZ2, DUX4, FAM22G, FAM90A1, GABBR2, GRM3, HMMR, HOXC4, KAAG1, KRIT1, KRTAP20-1, LOC392196, LOC441956, LOC650293, LTB4R, METTL7B, NEK2, OR11H12, OR2J3, OR2L8, OR2M1P, OR2T3, OR4F5, OR52A4, OR5211, PGA3, PHACTR3, PMP2, PRAMEF6, PSG1, SIGLEC10, SOX11, SPDYE1, SSX1, TCEB3B, TCFL5, TFAP2D, TSPY2, UGT2B10, UGT2B11, UGT2B28, WDR49, and WFDC5. Other biomarkers known in the relevant art may be used in combination with the biomarkers described herein.

B. Biomarker Panels

The biomarkers of the present invention can be used in diagnostic tests to assess, determine, and/or qualify (used interchangeably herein) prostate cancer status in a patient. The phrase “prostate cancer status” includes any distinguishable manifestation of the condition, including not having prostate cancer. For example, prostate cancer status includes, without limitation, the presence or absence of prostate cancer in a patient, the risk of developing prostate cancer, the stage or severity of prostate cancer, the progress of prostate cancer (e.g., progress of prostate cancer over time) and the effectiveness or response to treatment of prostate cancer (e.g., clinical follow up and surveillance of prostate cancer after treatment). Based on this status, further procedures may be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments, the biomarker panels of the present invention may show a statistical difference in different prostate cancer statuses of at least p<0.05, p<10⁻², p<10⁻³, p<10⁻⁴ or p<10⁻⁵. Diagnostic tests that use these biomarkers may show an ROC of at least 0.6, at least about 0.7, at least about 0.8, or at least about 0.9.

The biomarkers can be differentially present in UI (NC or non-prostate cancer) and prostate cancer, and, therefore, are useful in aiding in the determination of prostate cancer status. In certain embodiments, the biomarkers are measured in a patient sample using the methods described herein and compared, for example, to predefined biomarker levels/ratios and correlated to prostate cancer status. In particular embodiments, the measurement(s) may then be compared with a relevant diagnostic amount(s), cut-off(s), or multivariate model scores that distinguish a positive prostate cancer status from a negative prostate cancer status. The diagnostic amount(s) represents a measured amount of a biomarker(s) above which or below which a patient is classified as having a particular prostate cancer status. For example, if the biomarker(s) is/are up-regulated compared to normal during prostate cancer, then a measured amount(s) above the diagnostic cutoff(s) provides a diagnosis of prostate cancer. Alternatively, if the biomarker(s) is/are down-regulated during prostate cancer, then a measured amount(s) at or below the diagnostic cutoff(s) provides a diagnosis of non-prostate cancer. As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the amount of biomarkers in a statistically significant number of samples from patients with the different prostate cancer statuses, and drawing the cut-off to suit the desired levels of specificity and sensitivity.

In other embodiments, ratios of post-translationally modified biomarkers to the corresponding unmodified biomarkers are useful in aiding in the determination of prostate cancer status. In certain embodiments, the biomarker ratios are indicative of diagnosis. In other embodiments, a biomarker ratio can be compared to another biomarker ratio in the same sample or to a set of biomarker ratios from a control or reference sample.

Indeed, as the skilled artisan will appreciate there are many ways to use the measurements of two or more biomarkers in order to improve the diagnostic question under investigation. In a quite simple, but nonetheless often effective approach, a positive result is assumed if a sample is positive for at least one of the markers investigated.

Furthermore, in certain embodiments, the values measured for markers of a biomarker panel are mathematically combined and the combined value is correlated to the underlying diagnostic question. Biomarker values may be combined by any appropriate state of the art mathematical method. Well-known mathematical methods for correlating a marker combination to a disease status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate a biomarker combination of the present invention. In one embodiment, the method used in a correlating a biomarker combination of the present invention, e.g. to diagnose prostate cancer, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al.,12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

C. Determining Risk of Developing Prostate Cancer

In a specific embodiment, the present invention provides methods for determining the risk of developing prostate cancer in a patient. Biomarker percentages, ratios, amounts or patterns are characteristic of various risk states, e.g., high, medium or low. The risk of developing prostate cancer is determined by measuring the relevant biomarker(s) and then either submitting them to a classification algorithm or comparing them with a reference amount, i.e., a predefined level or pattern of biomarker(s) that is associated with the particular risk level.

D. Determining Prostate Cancer Severity

In another embodiment, the present invention provides methods for determining the severity of prostate cancer in a patient. Each grade or stage of prostate cancer likely has a characteristic level of a biomarker or relative levels/ratios of a set of biomarkers (a pattern or ratio). The severity of prostate cancer is determined by measuring the relevant biomarker(s) and then either submitting them to a classification algorithm or comparing them with a reference amount, i.e., a predefined level or pattern of biomarker(s) that is associated with the particular stage.

E. Determining Prostate Cancer Prognosis

In one embodiment, the present invention provides methods for determining the course of prostate cancer in a patient. Prostate cancer course refers to changes in prostate cancer status over time, including prostate cancer progression (worsening) and prostate cancer regression (improvement). Over time, the amount or relative amount (e.g., the pattern or ratio) of the biomarkers changes. For example, biomarker “X” may be increased with prostate cancer, while biomarker “Y” may be decreased with prostate cancer. Therefore, the trend of these biomarkers, either increased or decreased over time toward prostate cancer or non-prostate cancer indicates the course of the condition. Accordingly, this method involves measuring the level of one or more biomarkers in a patient at least two different time points, e.g., a first time and a second time, and comparing the change, if any. The course of prostate cancer is determined based on these comparisons.

F. Patient Management

In certain embodiments of the methods of qualifying prostate cancer status, the methods further comprise managing patient treatment based on the status. Such management includes the actions of the physician or clinician subsequent to determining prostate cancer status. For example, if a physician makes a diagnosis of prostate cancer, then a certain regime of monitoring would follow. An assessment of the course of prostate cancer using the methods of the present invention may then require a certain prostate cancer therapy regimen. Alternatively, a diagnosis of non-prostate cancer might be followed with further testing to determine a specific disease that the patient might be suffering from. Also, further tests may be called for if the diagnostic test gives an inconclusive result on prostate cancer status.

G. Determining Therapeutic Efficacy of Pharmaceutical Drug

In another embodiment, the present invention provides methods for determining the therapeutic efficacy of a pharmaceutical drug. These methods are useful in performing clinical trials of the drug, as well as monitoring the progress of a patient on the drug.

Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern, profile or ratio) of one or more of the biomarkers of the present invention may change toward a non-prostate cancer profile. Therefore, one can follow the course of one or more biomarkers in the patient during the course of treatment. Accordingly, this method involves measuring one or more biomarkers in a patient receiving drug therapy, and correlating the biomarker levels/ratios with the prostate cancer status of the patient (e.g., by comparison to predefined levels/ratios of the biomarkers that correspond to different prostate cancer statuses). One embodiment of this method involves determining the levels/ratios of one or more biomarkers for at least two different time points during a course of drug therapy, e.g., a first time and a second time, and comparing the change in levels/ratios of the biomarkers, if any. For example, the levels/ratios of one or more biomarkers can be measured before and after drug administration or at two different time points during drug administration. The effect of therapy is determined based on these comparisons. If a treatment is effective, then the level/ratio of one or more biomarkers will trend toward normal, while if treatment is ineffective, the level/ratio of one or more biomarkers will trend toward prostate cancer indications.

H. Generation of Classification Algorithms for Qualifying Prostate Cancer Status

In some embodiments, data that are generated using samples such as “known samples” can then be used to “train” a classification model. A “known sample” is a sample that has been pre-classified. The data that are used to form the classification model can be referred to as a “training data set.” The training data set that is used to form the classification model may comprise raw data or pre-processed data. Once trained, the classification model can recognize patterns in data generated using unknown samples. The classification model can then be used to classify the unknown samples into classes. This can be useful, for example, in predicting whether or not a particular biological sample is associated with a certain biological condition (e.g., diseased versus non-diseased).

Classification models can be formed using any suitable statistical classification or learning method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART), artificial neural networks such as back propagation networks, discriminant analyses (e.g., Bayesian classifier or Fischer analysis), logistic classifiers, and support vector classifiers (support vector machines).

Another supervised classification method is a recursive partitioning process. Recursive partitioning processes use recursive partitioning trees to classify data derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application Publication No. 2002/0193950 (Gavin et al. “Method or analyzing mass spectra”), U.S. Patent Application Publication No. 2003/0004402 (Hitt et al., “Process for discriminating between biological states based on hidden patterns from biological data”), and U.S. Patent Application Publication No. 2003/0055615 (Zhang and Zhang, “Systems and methods for processing biological expression data”).

The classification models can be formed on and used on any suitable digital computer. Suitable digital computers include micro, mini, or large computers using any standard or specialized operating system, such as a UNIX, Windows® or Linux™ based operating system. In embodiments utilizing a mass spectrometer, the digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including R, C, C++, visual basic, etc.

The learning algorithms described above are useful both for developing classification algorithms for the biomarkers already discovered, and for finding new biomarker biomarkers. The classification algorithms, in turn, form the base for diagnostic tests by providing diagnostic values (e.g., cut-off points) for biomarkers used singly or in combination.

### IV. Kits for the Detection of Prostate Cancer Biomarkers

In another aspect, the present invention provides kits for qualifying prostate cancer status, which kits are used to detect the biomarkers described herein. In a specific embodiment, the kit is provided as a PCR kit comprising primers that specifically bind to one or more of the biomarkers described herein. One of ordinary skill in the art can design primers the specifically bind and amplify the target biomarkers described herein including ACSM2A, BDH2, C19orf51, C8orf76, CGB5, CSMD3, DAZ2, DUX4, FAM22G, FAM90A1, GABBR2, GRM3, HMMR, HOXC4, KAAG1, KRIT1, KRTAP20-1, LOC392196, LOC441956, LOC650293, LTB4R, METTL7B, NEK2, OR11H12, OR2J3, OR2L8, OR2M1P, OR2T3, OR4F5, OR52A4, OR5211, PGA3, PHACTR3, PMP2, PRAMEF6, PSG1, SIGLEC10, SOX11, SPDYE1, SSX1, TCEB3B, TCFL5, TFAP2D, TSPY2, UGT2B10, UGT2B11, UGT2B28, WDR49, and WFDC5. The kit can further comprise substrates and other reagents necessary for conducting PCR (e.g., quantitative real-time PCR). The kit can be configured to conduct singleplex or multiplex PCR. The kit can further comprise instructions for carrying out the PCR reaction(s).

In another embodiment, the kit is provided as an ELISA kit comprising antibodies to the biomarker(s) of the present invention. In a specific embodiment, the antibodies specifically bind to a biomarker listed in Table 1.

The ELISA kit may comprise a solid support, such as a chip, microtiter plate (e.g., a 96-well plate), bead, or resin having biomarker capture reagents attached thereon. The kit may further comprise a means for detecting the biomarker(s), such as antibodies, and a secondary antibody-signal complex such as horseradish peroxidase (HRP)-conjugated goat anti-rabbit IgG antibody and tetramethyl benzidine (TMB) as a substrate for HRP.

The kit for qualifying prostate cancer status may be provided as an immuno-chromatography strip comprising a membrane on which the antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of blood serum.

In certain embodiments, a patient can be diagnosed by adding blood or blood serum from the patient to the kit and detecting the relevant biomarker(s) conjugated with antibodies, specifically, by a method which comprises the steps of: (i) collecting blood or blood serum from the patient; (ii) separating blood serum from the patient's blood; (iii) adding the blood serum from patient to a diagnostic kit; and, (iv) detecting the biomarker(s) conjugated with antibodies. In this method, the antibodies are brought into contact with the patient's blood. If the biomarkers are present in the sample, the antibodies will bind to the sample, or a portion thereof In other kit and diagnostic embodiments, blood or blood serum need not be collected from the patient (i.e., it is already collected). Moreover, in other embodiments, the sample may comprise a tissue sample or a clinical sample.

The kit can also comprise a washing solution or instructions for making a washing solution, in which the combination of the capture reagents and the washing solution allows capture of the biomarkers on the solid support for subsequent detection by, e.g., antibodies or mass spectrometry. In a further embodiment, a kit can comprise instructions for suitable operational parameters in the form of a label or separate insert. For example, the instructions may inform a consumer about how to collect the sample, how to wash the probe or the particular biomarkers to be detected, etc. In yet another embodiment, the kit can comprise one or more containers with biomarker samples, to be used as standard(s) for calibration.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

In particular embodiments, the genomic classifier comprises 49 genes specifically over-expressed by at least 4 fold in high grade prostate cancer which metastasizes within 5 years of complete surgical extirpation (Table 1). To obtain this classifier, genome wide expression profiling methods were performed on laser captured micro-dissected cells from formalin fixed paraffin embedded tissue of men undergoing radical prostatectomy for prostate cancer, simple prostatectomy for benign prostatic hyperplasia, and radical cystoprostatectomy for pathologically localized bladder cancer without prostate involvement or prostate cancer. Prostate cancer epithelial cells were laser captured from men with clinically localized prostate cancer which was either low grade (Gleason sum 6), high grade (Gleason sum 8-10) with men not experiencing metastasis following surgical treatment even with >10 years of follow up without adjuvant treatment, or high grade (Gleason sum 8-10) without lymph node involvement at prostatectomy but with men experiencing metastasis within 5 years of local treatment. Benign prostatic tissue was obtained by laser capture of cells from men undergoing simple prostatectomy for benign prostatic hyperplasia or undergoing radical cystoprostatectomy for bladder cancer (without neoadjuvant treatment) with no cancer identified in the prostate of the pathological specimen. “Normal urothelium” was obtained from radical cystoprostatectomy specimens of patients without carcinoma in situ and at areas distant from the bladder cancer lesion. Processing or gene expression data and statistical comparisons between gene expression signatures of the various groups was performed as described in Ross et al. 2011.

The primary goal was to identify genes which could be used to non-invasively detect prostate cancer with metastatic potential. Detection of disease in urine or serum samples implies the presence of other cell types. In addition, current technologies to identify small amounts of molecular material are more robust in identifying the presence rather than confirming the absence of an expressed gene or its product. Because of this, the identifier was based on genes which were over-expressed at least 4 fold in the prostate cancers of men who underwent radical prostatectomy for high grade disease and developed distant metastasis within 5 years of local treatment as compared to cells from all other profiled categories (low grade prostate cancer, high grade prostate cancer without metastatic potential, normal prostate epithelium, benign prostatic hyperplasia and urothelium). To be included in the classifiers, adjusted p-values of <0.01 for the comparison were required (Table 1).

The gene classifier can be used with standard technologies (i.e., quantitative, multiplexed real time PCR) to identify clinically significant and highly aggressive prostate cancer in urine, serum and semen. In addition, this classifier can be used to sub stratify prostate cancer even following biopsy or treatment to aid in the section of local and possible adjuvant therapies.

TABLE 1
Genomic Classifiers of High Grade Prostate Cancer with Metastatic Potential

| Gene | Fold Over- | adjusted | ENTREZ | |
|---|---|---|---|---|
| Symbol | expression | P. Val | ID | Gene Name |
| ACSM2A | 5.326 | 6.41E−12 | 123876 | acyl-CoA synthetase medium-chain family member 2A |
| BDH2 | 5.154 | 1.51E−12 | 56898 | 3-hydroxybutyrate dehydrogenase type 2 |
| C19orf51 | 4.532 | 0.0008648 | 352909 | chromosome 19 open reading frame 51 |
| C8orf76 | 5.774 | 4.21E−07 | 84933 | chromosome 8 open reading frame 76 |
| CGB5 | 4.196 | 0.002306 | 93659 | chorionic gonadotropin beta polypeptide 5 |
| CSMD3 | 5.074 | 6.52E−06 | 114788 | CUB and Sushi multiple domains 3 |
| DAZ2 | 6.052 | 4.63E−06 | 57055 | deleted in azoospermia 2 |
| DUX4 | 5.572 | 8.23E−05 | 22947 | double homeobox 4 |
| FAM22G | 4.52 | 2.28E−07 | 441457 | family with sequence similarity 22 member G |
| FAM90A1 | 5.448 | 9.86E−06 | 55138 | family with sequence similarity 90 member A1 |
| GABBR2 | 6.988 | 5.18E−13 | 9568 | gamma-aminobutyric acid (GABA) B receptor 2 |
| GRM3 | 6.832 | 1.37E−06 | 2913 | glutamate receptor metabotropic 3 |
| HMMR | 4.592 | 4.95E−06 | 3161 | hyaluronan-mediated motility receptor (RHAMM) |
| HOXC4 | 4.388 | 1.76E−05 | 3221 | homeobox C4 |
| KAAG1 | 4.232 | 0.001041 | 353219 | kidney associated antigen 1 |
| KRIT1 | 7.44 | 3.27E−07 | 889 | KRIT1 ankyrin repeat containing |
| KRTAP20-1 | 4.756 | 1.27E−09 | 337975 | keratin associated protein 20-1 |
| LOC392196 | 5.394 | 1.98E−11 | 392196 | deubiquitinating enzyme 3 pseudogene |
| LOC441956 | 5.45 | 6.23E−08 | 441956 | similar to cDNA sequence BC021523 |
| LOC650293 | 4.838 | 0.0004872 | 650293 | seven transmembrane helix receptor |
| LTB4R | 4.188 | 0.004217 | 1241 | leukotriene B4 receptor |
| METTL7B | 6.846 | 3.91E−15 | 196410 | methyltransferase like 7B |
| NEK2 | 4.538 | 0.003043 | 4751 | NIMA (never in mitosis gene a)-related kinase 2 |
| OR11H12 | 4.638 | 0.0003957 | 440153 | olfactory receptor family 11 subfamily H member 12 |
| OR2J3 | 4.486 | 0.0004773 | 442186 | olfactory receptor family 2 subfamily J member 3 |
| OR2L8 | 5.38 | 3.31E−07 | 391190 | olfactory receptor family 2 subfamily L member 8 |
| OR2M1P | 4.108 | 0.0002209 | 388762 | olfactory receptor family 2 subfamily M member 1 pseudogene |
| OR2T3 | 4.3 | 0.0004847 | 343173 | olfactory receptor family 2 subfamily T member 3 |
| OR4F5 | 4.65 | 3.52E−05 | 79501 | olfactory receptor family 4 subfamily F member 5 |
| OR52A4 | 4.064 | 4.63E−07 | 390053 | olfactory receptor family 52 subfamily A member 4 |
| OR52I1 | 4.504 | 2.30E−13 | 390037 | olfactory receptor family 52 subfamily I member 1 |
| PGA3 | 4.462 | 3.36E−16 | 643834 | pepsinogen 3 group I (pepsinogen A) |
| PHACTR3 | 5.688 | 4.23E−06 | 116154 | phosphatase and actin regulator 3 |
| PMP2 | 4.6 | 9.13E−05 | 5375 | peripheral myelin protein 2 |
| PRAMEF6 | 5.268 | 3.16E−05 | 440561 | PRAME family member 6 |
| PSG1 | 6.024 | 1.86E−06 | 5669 | pregnancy specific beta-1-glycoprotein 1 |
| SIGLEC10 | 5.188 | 3.30E−05 | 89790 | sialic acid binding Ig-like lectin 10 |
| SOX11 | 5.418 | 3.28E−05 | 6664 | SRY (sex determining region Y)-box 11 |
| SPDYE1 | 5.168 | 2.16E−06 | 285955 | speedy homolog E1 (Xenopus laevis) |
| SSX1 | 4.214 | 0.002396 | 6756 | synovial sarcoma X breakpoint 1 |
| TCEB3B | 5.16 | 0.002127 | 51224 | transcription elongation factor B polypeptide 3B (elongin A2) |
| TCFL5 | 4.522 | 0.001016 | 10732 | transcription factor-like 5 (basic helix-loop-helix) |
| TFAP2D | 5.726 | 3.77E−16 | 83741 | transcription factor AP-2 delta (activating enhancer binding protein 2 delta) |
| TSPY2 | 5.866 | 0.000381 | 64591 | testis specific protein Y-linked 2 |
| UGT2B10 | 4.484 | 0.0005598 | 7365 | UDP glucuronosyltransferase 2 family polypeptide B10 |
| UGT2B11 | 5.124 | 7.29E−05 | 10720 | UDP glucuronosyltransferase 2 family polypeptide B11 |
| UGT2B28 | 5.586 | 6.00E−06 | 54490 | UDP glucuronosyltransferase 2 family polypeptide B28 |
| WDR49 | 4.296 | 2.85E−05 | 151790 | WD repeat domain 49 |
| WFDC5 | 5.75 | 4.81E−05 | 149708 | WAP four-disulfide core domain 5 |

### REFERENCES

1. Ross A E, Marchionni L, Vuica-Ross M, et al. Gene expression pathways of high grade localized prostate cancer. Prostate 2011.

2. Andriole G L, Crawford E D, Grubb R L, 3rd, et al: Mortality results from a randomized prostate-cancer screening trial. N Engl J Med 360:1310-9, 2009.

3. Schroder F H, Hugosson J, Roobol M J, et al: Screening and prostate-cancer mortality in a randomized European study. N Engl J Med 360:1320-8, 2009.

4. Tosoian J J, Loeb S, Kettermann A, Landis P, Elliot D J, Epstein J I, Partin A W, Carter H B, Sokoll U. Accuracy of PCA3 measurement in predicting short-term biopsy progression in an active surveillance program. J Urol. 2010 February; 183(2):534-8.

5. Cuzick J, Swanson G P, Fisher G, Brothman A R, Berney D M, Reid J E, Mesher D, Speights V O, Stankiewicz E, Foster C S, Møller H, Scardino P, Warren J D, Park J, Younus A, Flake D D 2nd, Wagner S, Gutin A, Lanchbury J S, Stone S; Transatlantic Prostate Group. Prognostic value of an RNA expression signature derived from cell cycle proliferation genes in patients with prostate cancer: a retrospective study. Lancet Oncol. 2011 March; 12(3):245-55.

6. Laxman B, Morris D S, Yu J, et al: A first-generation multiplex biomarker analysis of urine for the early detection of prostate cancer. Cancer Res 68:645-9, 2008.

1. A method for diagnosing high grade prostate cancer with metastatic potential in a patient comprising the steps of:
a. obtaining a biological sample from the patient;
b. quantitating the biomarker expression levels of one or more of ACSM2A, BDH2, C19orf51, C8orf76, CGB5, CSMD3, DAZ2, DUX4, FAM22G, FAM90A1, GABBR2, GRM3, HMMR, HOXC4, KAAG1, KRIT1, KRTAP20-1, LOC392196, LOC441956, LOC650293, LTB4R, METTL7B, NEK2, OR11H12, OR2J3, OR2L8, OR2M1P, OR2T3, OR4F5, OR52A4, OR5211, PGA3, PHACTR3, PMP2, PRAMEF6, PSG1, SIGLEC10, SOX11, SPDYE1, SSX1, TCEB3B, TCFL5, TFAP2D, TSPY2, UGT2B10, UGT2B11, UGT2B28, WDR49, and WFDC5;
c. comparing the levels of the one or more biomarkers with reference levels of the one or more biomarkers that correlate to a patient not having prostate cancer with metastatic potential; and
d. identifying the patient as having prostate cancer with metastatic potential if the quantitated amounts of the one or more biomarkers is increased compared to the reference levels.

2. The method of claim 1, wherein the sample is blood, plasma, serum, urine, stool or semen.

3. The method of claim 2, wherein the sample is a urine sample.

4. The method of claim 2, wherein the sample is a semen sample.

5. The method of claim 2, wherein the sample is a serum sample.

6. The method of claim 1, wherein the quantitation step comprises performing multiplex quantitative real-time polymerase chain reaction.

7. The method of claim 1, wherein the patient not having prostate cancer with metastatic potential comprises one or more of patients with low grade prostate cancer, high grade prostate cancer without metastatic potential, normal prostate epithelium, benign prostate hyperplasia and benign urothelium.

8. The method of claim 1, wherein the levels of the one or more biomarkers from the patient sample are increased at least 4-fold as compared to reference levels of the same biomarkers.

9. A method for diagnosing high grade prostate cancer with metastatic potential in a patient comprising the steps of:
a. obtaining a biological sample from the patient;
b. subjecting the sample to an assay for detecting expression of one or more of ACSM2A, BDH2, C19orf51, C8orf76, CGB5, CSMD3, DAZ2, DUX4, FAM22G, FAM90A1, GABBR2, GRM3, HMMR, HOXC4, KAAG1, KRIT1, KRTAP20-1, LOC392196, LOC441956, LOC650293, LTB4R, METTL7B, NEK2, OR11H12, OR2J3, OR2L8, OR2M1P, OR2T3, OR4F5, OR52A4, OR52I1, PGA3, PHACTR3, PMP2, PRAMEF6, PSG1, SIGLEC10, SOX11, SPDYE1, SSX1, TCEB3B, TCFL5, TFAP2D, TSPY2, UGT2B10, UGT2B11, UGT2B28, WDR49, and WFDC5; and
c. comparing the levels of the one or more biomarkers with predefined levels of the same biomarkers that correlate to a patient having high grade prostate cancer with metastatic potential and predefined levels of the same biomarkers that correlate to a patient not having high grade prostate cancer with metastatic potential, wherein a correlation to one of the predefined levels provides the diagnosis.

10. The method of claim 9, wherein the sample is blood, plasma, serum, urine, stool or semen.

11. The method of claim 10, wherein the sample is a urine sample.

12. The method of claim 10, wherein the sample is a semen sample.

13. The method of claim 10, wherein the sample is a serum sample.

14. The method of claim 9, wherein the assay for detecting expression is an immunoassay.

15. The method of claim 9, wherein the assay for detecting expression is mass spectrometry.

16. The method of claim 15, wherein the mass spectrometry is multiple reaction monitoring mass spectrometry (MRM-MS).

17. A method for treating a patient suspected of having or likely to develop high grade prostate cancer with metastatic potential comprising the steps of:
a. obtaining a biological sample from the patient;
b. quantitating the biomarker expression levels of one or more of ACSM2A, BDH2, C19orf51, C8orf76, CGB5, CSMD3, DAZ2, DUX4, FAM22G, FAM90A1, GABBR2, GRM3, HMMR, HOXC4, KAAG1, KRIT1, KRTAP20-1, LOC392196, LOC441956, LOC650293, LTB4R, METTL7B, NEK2, OR11H12, OR2J3, OR2L8, OR2M1P, OR2T3, OR4F5, OR52A4, OR5211, PGA3, PHACTR3, PMP2, PRAMEF6, PSG1, SIGLEC10, SOX11, SPDYE1, SSX1, TCEB3B, TCFL5, TFAP2D, TSPY2, UGT2B10, UGT2B11, UGT2B28, WDR49, and WFDC5;
c. comparing the levels of the one or more biomarkers with reference levels of the one or more biomarkers that correlate to a patient not having prostate cancer with metastatic potential;
d. identifying the patient as having or likely to develop prostate cancer with metastatic potential if the quantitated amounts of the one or more biomarkers is increased compared to the reference levels; and
e. performing prostatectomy on the patient.

18. The method of claim 17, wherein the sample is blood, plasma, serum, urine, stool or semen.

19. A method for diagnosing high grade prostate cancer with metastatic potential in a patient comprising the steps of:
a. obtaining a sample from a patient suspected of having prostate cancer;
b. quantitating the amount of the one or more biomarkers listed in Table 1, wherein the quantitating step comprises (i) contacting the sample with a set of primers capable of amplifying one or more of the biomarkers listed in Table 1; and (ii) amplifying the one or more biomarkers listed in Table 1;
c. comparing the quantitated amounts of the one or more biomarkers listed in Table 1 to a reference level; and
d. identifying the patient as having prostate cancer if the quantitated amounts of the one or more biomarkers is increased compared to the reference level

20. A prostate cancer genomic classifier comprising one or more biomarkers listed in Table 1.
